(19) United States
(12) Patent Application Publication
(10) Pub. No.: US 2012/0093907 A1
(43) Pub. Date: Apr. 19, 2012

(54) COMPOSITE OF SILVER NANOPARTICLE AND LAYERED INORGANIC CLAY FOR INHIBITING GROWTH OF SILVER-RESISTANT BACTERIA

(75) Inventors: Jiang-Jen Lin, Taipei (TW); Hong-Lin Su, Taipei (TW); I-Chuan Pao, Taipei (TW); Siou-Hong Lin, Taipei (TW)

(73) Assignee: NATIONAL TAIWAN UNIVERSITY, Taipei (TW)

(21) Appl. No.: 13/102,017

(22) Filed: May 5, 2011

(30) Foreign Application Priority Data
Oct. 15, 2010 (TW) 099135333

(51) Int. Cl. A01N 25/26; A01P 15/00; A01N 59/16; B82Y 5/00

(52) U.S. Cl. 424/409; 424/618; 977/773

(57) ABSTRACT

The present invention provides a composite of spherical silver nanoparticles and layered inorganic clay. This composite can effectively inhibit the growth of silver-resistant bacteria. The layered inorganic clay serves as carriers of the silver nanoparticles and disperses them. The composite has a particle size of about 5 nm to 100 nm. The silver nanoparticles can be dispersed in an organic solvent or water.

### BACKGROUND OF THE INVENTION

1. Field of the Invention

The present invention relates to a composite of silver nanoparticles (AgNPs) and layered inorganic clay for inhibiting growth of bacteria, particularly silver-resistant bacteria. The composite can be used in biomedical applications, for example, controlling nosocomial infection and treatments of burning.

2. Related Prior Arts

It is well known that silver nanoparticles can effectively inhibit growth of most bacteria. One of the mechanisms is that silver ions dissociated from silver nanoparticles can enter bacteria through the cell walls/membranes of the bacteria to combine with the proteins or DNA of the bacteria. As a result, physiological functions of the bacteria are destroyed and thus growth of the bacteria is inhibited.

However, silver-resistant bacteria possess a special protein on cell membranes capable of delivering silver ions outside the cells and thus they are not destroyed by the silver ions. For example, Escherichia coli strain J53 pMG101 can survive impacts of silver ions of more than 1 mM. That is, to kill silver-resistant bacteria, it's required to provide silver ions of higher concentrations which however are cytotoxic.

To solve the above problems, the present invention provides a composite capable of inhibiting growth of silver-resistant bacteria at lower concentrations of silver ions without cytotoxicity.

### SUMMARY OF THE INVENTION

The object of the present invention is to provide a composite of silver nanoparticles (AgNPs) and inorganic clay, which can effectively inhibit the growth of silver-resistant bacteria at lower concentrations of silver ions.

This composite includes AgNPs and layered inorganic clay, wherein the layered inorganic clay has an aspect ratio (width/thickness ratio) of about 10 to 100,000 and serves as carriers of the AgNPs to disperse the AgNPs nanoparticles. The composite has a size of about 5 nm to 100 nm, and preferably from 20 nm to 30 nm. The ratio of the ionic equivalent of the AgNPs to the cationic exchange equivalent (CEC) of the layered inorganic clay (Ag⁺/CEC) is from 0.1/1 to 200/1, and preferably from 0.5/1 to 2/1. The AgNPs/clay weight ratio is from 1/99 to 99/1, and preferably from 1/99 to 10/90.

The composite is preferably used for inhibiting the growth of multi-silver-resistant bacteria, for example, silver-resistant Acinetobacter baumannii and Escherichia coli.

The layered inorganic clay preferably has an aspect ratio of about 100 to 1,000.

The layered inorganic clay can be bentonite, laponite, montmorillonite, synthetic mica, kaolin, talc, attapulgite clay, vermiculite or double hydroxide (LDH) nanoparticles, preferably having a structure with silicon-tetrahedron: aluminum-octahedron of about 2:1. More preferably, the layered inorganic clay is silicate platelets or hectorite nanoparticles.

In the composite, the AgNPs/clay weight ratio is preferably about 1/99 to 20/80, and more preferably 3/97 to 10/90.

The composite can further include a solvent in which the composite is present in a concentration of 0.0001 wt % to 10.0 wt %, preferably 0.001 wt % to 1.0 wt %, and more preferably 0.01 wt % to 0.2 wt %.

In the composite, the CEC of the layered inorganic clay is about 0.1 mequiv/g to 5.0 mequiv/g.

In the composite, the ratio of Ag⁺/CEC is preferably about 0.1/1 to 10/1, and more preferably 0.5/1 to 2/1.

### BRIEF DESCRIPTION OF THE DRAWINGS

FIG. 1 shows the growth of Acinetobacter baumannii (AB) in the agarose gel including silver nitrate in various concentrations.

FIG. 2 shows the growth of Escherichia coli in the agarose gel including silver nitrate in various concentrations.

FIG. 3 shows the growth of Acinetobacter baumannii in the agarose gel including AgNP/SWN in various concentrations.

FIG. 4 shows Escherichia coli growing in the agarose gel including AgNP/SWN in various concentrations.

FIG. 5 shows the growth of Acinetobacter baumannii in the agarose gel including AgNP/NSP in various ratios.

FIG. 6 shows the growth of Escherichia coli in the agarose gel including AgNP/NSP in various concentrations.

FIG. 7 shows the percentages of the dead cells.

FIG. 8 shows the percentages of the radicals generated by bacteria.

### DETAILED DESCRIPTION OF THE PREFERRED EMBODIMENTS

The materials used in the preferred embodiments and applications of the present invention include:

1. Nanosilicate platelets (NSP): cation exchange capacity (CEC)=1.20 mequiv/g; having a single-layered or dual-layered structure in water; isoelectric point (IE)=pH 6.4; almost 100% inorganic; available by exfoliating montmorillonite (Na⁺-MMT); as described in: U.S. Pat. No. 7,125,916, U.S. Pat. Nos. 7,094,815, 7,022,299, and 7,442,728 or U.S. Publication No. 2006-0287413-A1.

2. Hectorite: Product of CO-OP Chemical Co. (Japan), SWN®, was used, synthetic layered silicate clay, a kind of bentonite, cationic exchange capacity (CEC)=0.67 mequiv/g.

3. AgNO₃: Used for exchanging or replacing Na⁺ between layers of the clay and for providing silver ions to be reduced to Ag nanoparticles (AgNPs).

4. Methanol: CH₃OH, 95%, a weak reducing agent, used to reduce the silver ions to AgNPs at 30˜150° C. .

5. Ethylene glycol (EG): C₂H₄(OH)₂, a weak reducing agent, used to reduce the silver ions to AgNPs at 30˜150° C. .

6. Microorganisms:

(1) Acinetobacter baumannii: Including ordinary, multidrug-resistant and silver-resistant strains, provided by Dr. Huang Chieh-Chen of National Chung Hsing University, Department of Life Sciences, Taiwan.

(2) Escherichia coli: Isolated from wild colonies and used as type culture of Gram-negative bacteria; provided by Dr. Lin Chun-Hung of Animal Technology Institute Taiwan.

(3) Escherichia coli J53: Used as control groups to the silver-resistant strain J53pMG101, having no silver-resistant plasmid pMG101, provided by Prof. C. M. Che, Department of Chemistry, The University of Hong Kong.

(4) Silver-resistant Escherichia coli J53pMG101: Having silver-resistant plasmid pMG101, provided by Dr. Anne O. Summers, Department of Microbiology, The University of Georgia, Athens, US.

7. Preparation of the standard suspensions of bacteria

The suspensions of bacteria cultured overnight were added into a fresh Luria-Bertani (LB) liquid media at a volume ratio of 1/100 for culturing for about three hours. Absorbance (OD₆₀₀) of the suspensions of bacteria after culturing were determined with a spectrophotometer, and the suspensions having OD₆₀₀ values ranging between 0.4 to 0.6 were selected as the standard suspensions of bacteria.

In the present invention, the preferred natural and synthetic clay include:

1. Synthetic fluorine mica: mica, product of CO-OP Chemical Co. (Japan), code number SOMASIF ME-100, with cationic exchange capacity (CEC)=1.20 mequiv/g.

2. Laponite: Synthetic layered silicate clay with cationic exchange capacity (CEC)=0.69 mequiv/g.

3. [M^{II}₁₋ₓM^{III}ₓ(OH)₂]ᵢₙₜᵣₐ[Aⁿ⁻·nH₂O]ᵢₙₜₑᵣ: Synthetic layered double hydroxide with ionic exchange capacity in the range of 2.0 to 4.0 mequiv./g, M^{II} is a two-valence metal ion, for example, Mg, Ni, Cu and Zn; M^{III} is a three-valence metal ion, for example, Al, Cr, Fe, V and Ga; Aⁿ⁻ is an anion, for example, CO₃²⁻, NO₃⁻.

The procedure of producing the AgNPs/clay composite were as follows:

### (1) AgNP/SWN

First, the SWN solution (1 wt %) and the AgNO₃ solution (1 wt %) were prepared. The AgNO_{3(aq)} (3.4143 g) was then slowly added into the SWN solution (30 g) so that the Ag⁺/CEC equivalent ratio was 1.0/1.0 and the Ag⁺/SWN weight ratio was about 7/93. The solution immediately bacame light yellow. Into this solution, methanol (MeOH, about 6˜8 mL) was added and the solution remained in light yellow. By means of ultrasonic mixing and water bath at 70˜80° C., the reaction began and the color changed. After vibration, the product AgNP/SWN was achieved. The AgNP/SWN solution was diluted to 60 μM (0.01 wt %), 600 μM (0.1 wt %) and 1.2 mM (0.2 wt %), respectively, for tests of inhibiting bacterial growth.

### (2) AgNP/NSP

First, the NSP solution (1 wt %) and the AgNO₃ solution (1 wt %) were prepared. The AgNO_{3(aq)} (3.5160 g) was then slowly added into the NSP solution (30 g) so that the Ag⁺/CEC equivalent ratio was 1.0/1.0 and the Ag⁺/NSP weight ratio was about 7/93. The Na⁺ ions between the clay layers were replaced with the Ag⁺ ions and the solution turned into a cream color. Into this solution, ethylen glycol (EG, about 0.1˜5 mL) was added and the solution was still in cream color. By means of ultrasonic mixing and water bath at 40˜80° C., the reaction began and the color changed. After vibration, the product AgNP/NSP was obtained. The AgNP/NSP solution was diluted to 60 μM (0.01 wt %), 600 μM (0.1 wt %) and 1.2 mM (0.2 wt %), respectively, for tests of inhibiting bacterial growth.

In the above AgNP/clay composites, clay served as carriers for adsorbing the AgNPs to kill ordinary bacteria and multidrug-resistant bacteria. The AgNPs had a particle size of about 20 to 30 nm. Measured with inductively coupled plasma-mass spectrometry (ICP-MS), the silver ions in the AgNP/clay composite solution (0.1 wt %) had a concentration of about 120 to 190 ppb.

In the present invention, the tests of inhibiting bacterial growth were performed by adding the water solutions of silver nitrate, AgNP/SWN or AgNP/NSP of different ratios into the uncoagulated LB solid culture media to prepare 100 mm LB solid cultere media of different concentrations.

The standard suspensions of bacteria (each 100 μl) were spread on the LB solid media including silver nitrate of different concentrations with sterilized glass beads to culture at 37° C. for 16 hours. The numbers of colonies were determined by dividing the plate into 8 or 16 areas wherein one area was selected to count the colonies thereon. The total number of colonies was obtained by multiplying the number of colonies on the selected area with the number of the areas. Results were as follows, wherein the mock group without treatment was relatively set as 100% and the colony ratios (%) could be used to estimae the inhibition effects (=100%−the colony ratio).

### 1. Solid Media Including Silver Nitrate

1.1 Acinetobacter baumannii

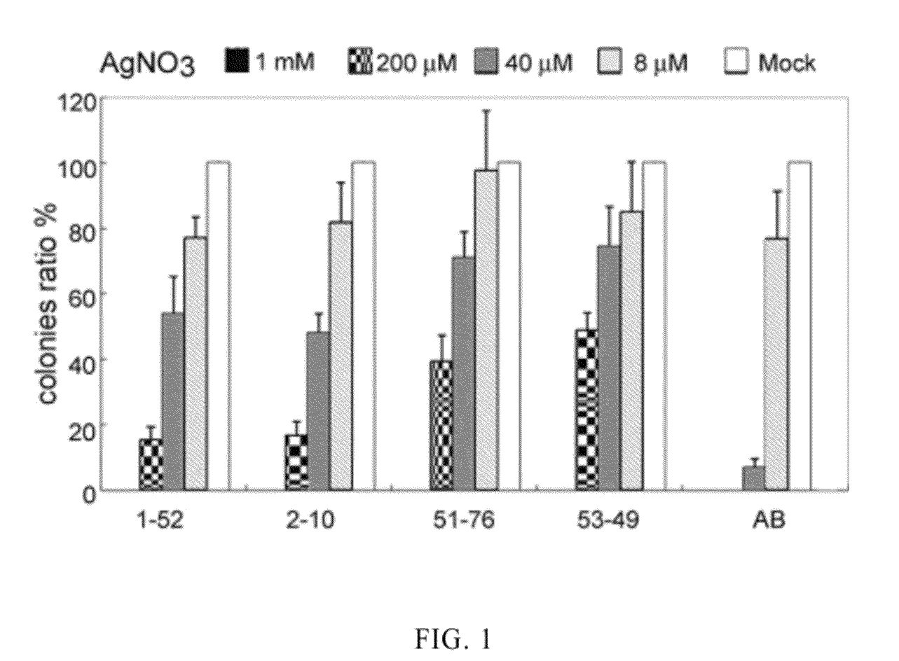

As shown in FIG. 1, for Acinetobacter baumannii (AB) without drug-resistance, growth could not be effectively inhibited in silver nitrate (8 μM), 90% could be inhibited in silver nitrate (40 μM) and all could be inhibited in silver nitrate (200 μM).

For the silver-resistant Acinetobacter baumannii strains (1-52, 2-10, 51-76, 53-49), only 50˜80% were inhibited in silver nitrate (200μ). The concentration of silver ions had to be as high as 1 mM for all of the bacteria to be inhibited.

1.2 Escherichia coli

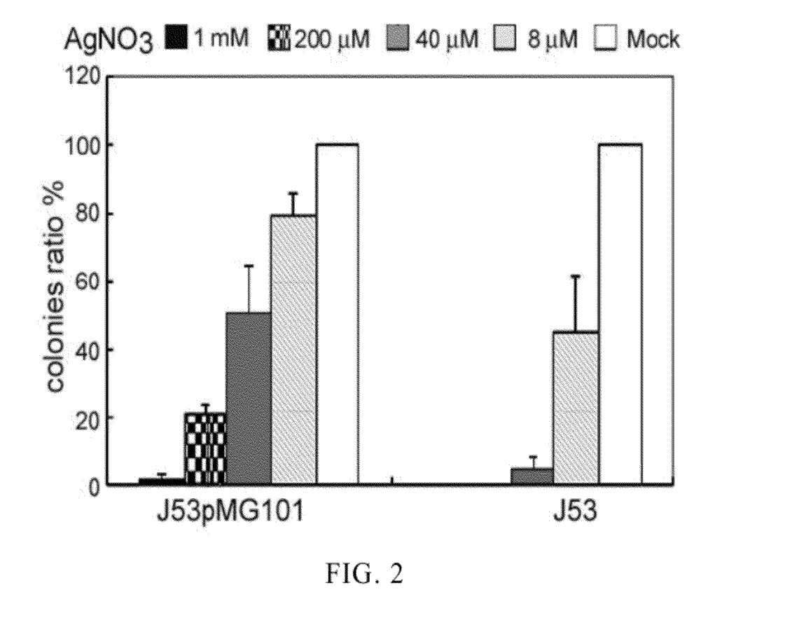

As shown in FIG. 2, for Escherichia coli (J53 strain) without drug-resistance, growth could not be effectively inhibited in silver nitrate (8 μM), 90% could be inhibited in silver nitrate (40 μM) and all could be inhibited in silver nitrate (200 μM).

For the silver-resistant Escherichia coli (J53pMG101), only about 80% were inhibited in silver nitrate (200 μM). The concentration of silver ions had to be as high as 1 mM for almost all of the bacteria to be inhibited.

### 2. Solid Media Including AgNP/SWN

2.1 Acinetobacter baumannii

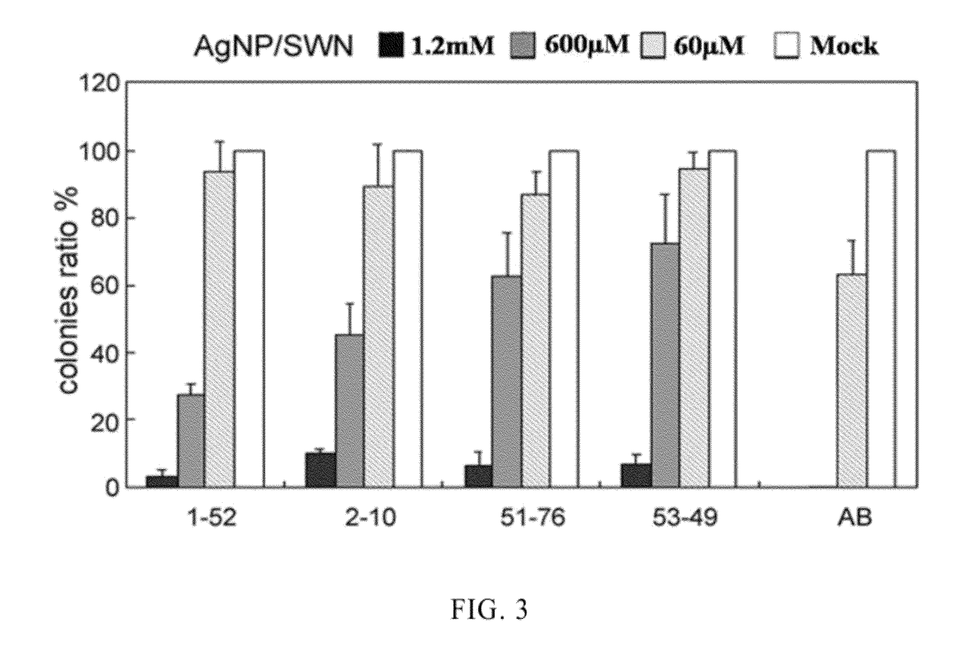

As shown in FIG. 3, for Acinetobacter baumannii (AB) without drug-resistance, growth could not be effectively inhibited in AgNP/SWN (60 μM) and all could be inhibited in AgNP/SWN (600 μM).

For the silver-resistant Acinetobacter baumannii strains (1-52, 2-10, 51-76, 53-49), only 50˜80% were inhibited in AgNP/SWN (600 μM). Even in AgNP/SWN (1.2 mM), about 5% of the bacteria could still be live.

2.2 Escherichia coli

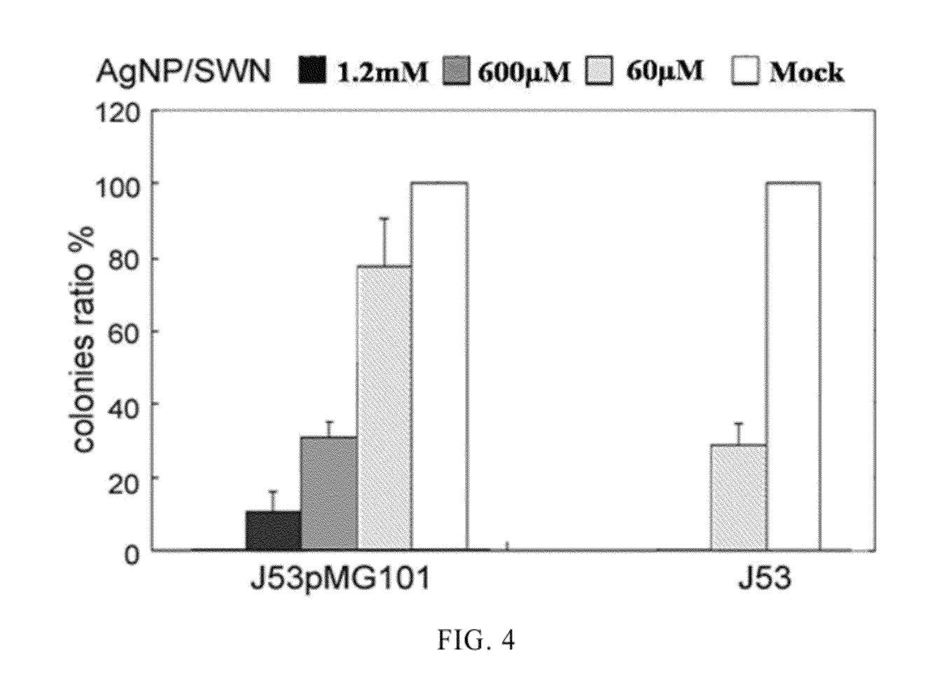

As shown in FIG. 4, for Escherichia coli (J53 strain) without drug-resistance, growth could not be effectively inhibited in AgNP/SWN (60 μM) and all could be inhibited in AgNP/SWN (600 μM).

For the silver-resistant Escherichia coli (J53pMG 101 strain), only 50˜80% were inhibited in AgNP/SWN (600 μM). Even in AgNP/SWN (1.2 mM), 10% of the bacteria could still be live.

### 3. Solid Media Including AgNP/NSP

3.1 Acinetobacter baumannii

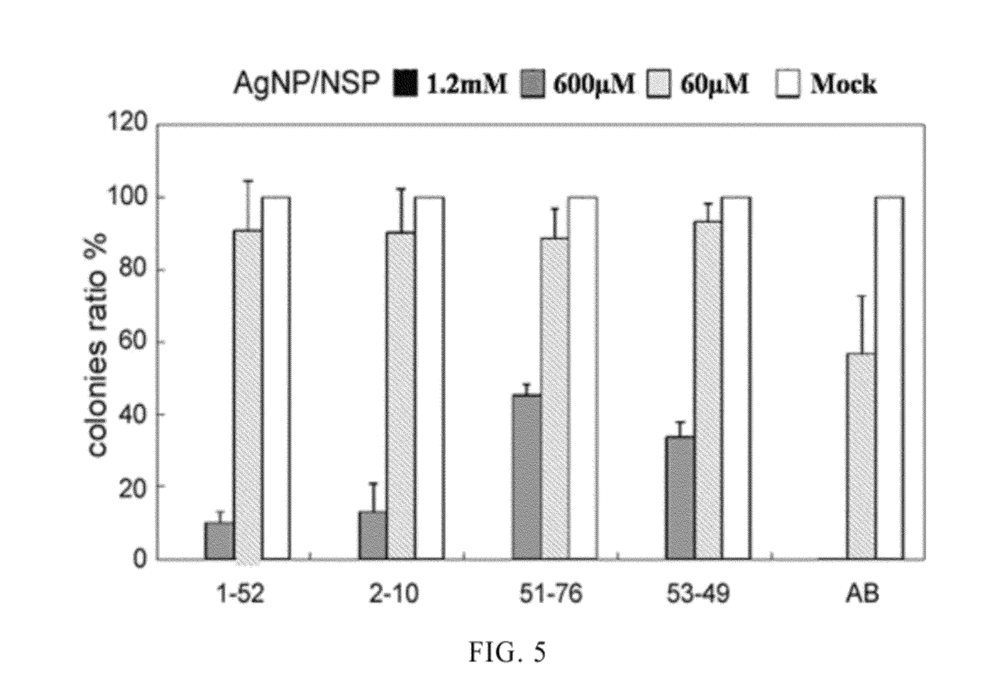

As shown in FIG. 5, for Acinetobacter baumannii (AB) without drug-resistance, growth could not be effectively inhibited in AgNP/NSP (60 μM) and all could be inhibited in AgNP/NSP (600 μM).

For the silver-resistant Acinetobacter baumannii strains (1-52, 2-10, 51-76, 53-49), only 50˜80% were inhibited in AgNP/NSP (600 μM). The bacteria could be completely inhibited in AgNP/NSP (1.2 mM), which indicated that AgNP/NSP performed better than AgNP/SWN. The reason could be that the single-layered NSP provides larger contact area than SWN constructed with 8 to 10 layers.

3.2 Escherichia coli

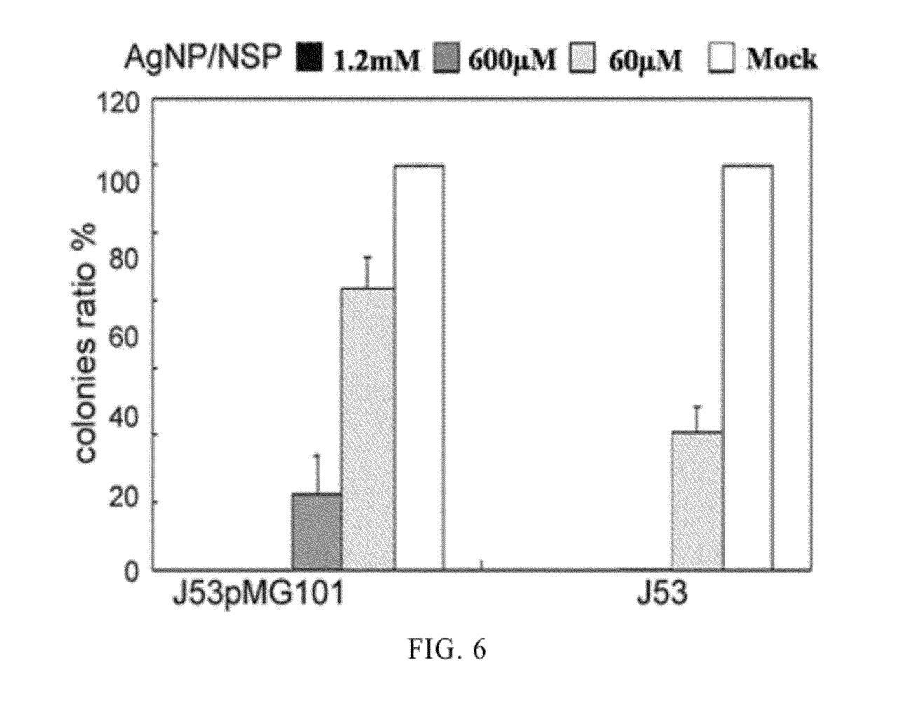

As shown in FIG. 6, for Escherichia coli (J53 strain) without drug-resistance, growth could not be effectively inhibited in the agarose gel including AgNP/NSP (60 μM) and all could be inhibited in AgNP/SWN (600 μM).

For the silver-resistant Escherichia coli (J53pMG101), only about 80% were inhibited in AgNP/NSP (600 μM). The bacteria could be completely inhibited in AgNP/NSP (1.2 mM), which indicated that AgNP/NSP performed better than AgNP/SWN. The reason could be that the single-layered NSP provides larger contact area than SWN constructed with 8 to 10 layers.

According to the analysis of the composite (600 μM, 0.1 wt %), the silver ions were present in a concentration of only 150 ppb (about 1˜1.5 μM) in the upper clear liquid. Since the silver ions of such concentrations could not kill bacteria, the composite of the present invnetion could not inhibit growth of bacteria through the dissociated silver ions. Therefore, the bacteria must have been killed through lots of radicals which could destroy cell membranes thereof.

The above mechanism could be verified by the following methods:

### 1. Determining the Live/Dead Cells

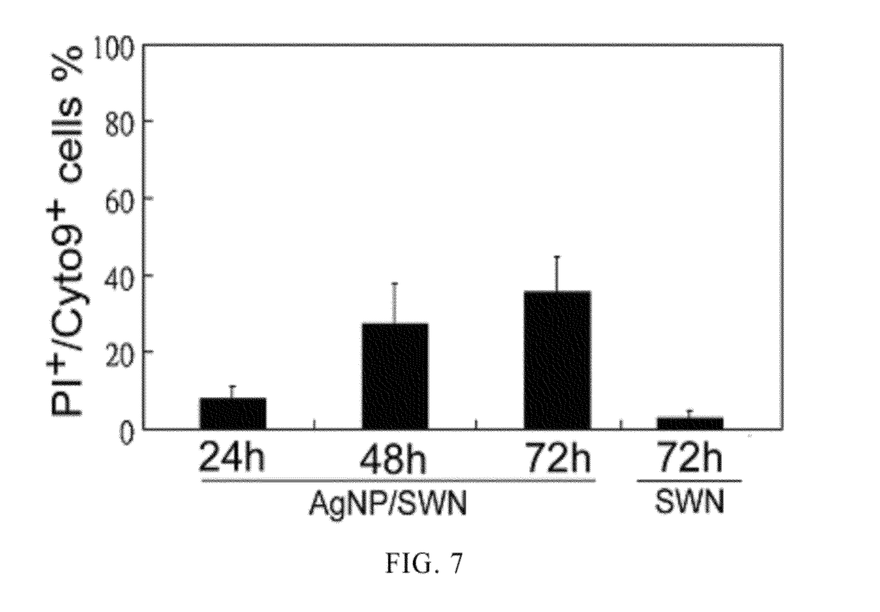

LIVE/DEAD BacLight kit (Invitrogen) was used to determine whether a cell is live or dead. All cells could be stained with cyto9, but only the damaged cells of bacteria could be stained with propidium iodide (PI). By combing these two stain reagents, the live cells could be distinguished from the dead. The bacteria were stained at room temperature with slow vibration, at about 50 rpm. At certain intervals, the cells were monitored with a microscope (oil immersion). FIG. 7 shows the percentages of the dead cells among all the bacteria cells: the bacteria treated with AgNP/SWN after 72 hours were about 38±6.8% dead, and the bacteria treated with SWN were about 10% dead.

### 2. Determining the Radicals

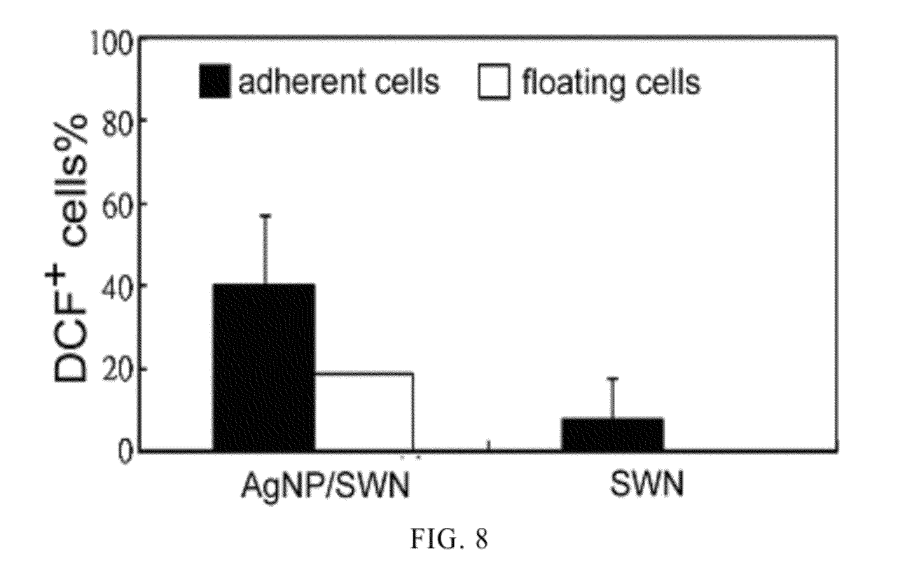

When the cells generated radicals, for example, reactive oxygen species (ROS), DCFH-DA (2′,7′-dichlorofluorescin-diacetate) would be oxidized to DCF (dichlorofluorescin) and emit fluorescent light. Brightness of the fluorescent light was proportional to the amount of the radicals. In the present invention, DCFH-DA (10M) was applied to the bacteria which were observed under microscope at the 0.5th, 1st and 2nd hours. Percentages (PI⁺/Cyto9⁺ Cells %) of the bacteria generating fluorescent light to the total bacteria could be estimated. Escherichia coli strains treated with AgNP/SWN and SWN were monitored. The microscope images indicated that the strains emit more green fuorecent light after being treated with SWN or AgNP/SWN (300 μM, 0.05 wt %) for 2 hours; and the strains emit more red fuorecent light after being treated with SWN or AgNP/SWN (600 μM, 0.1 wt %) for 24 and 48 hours. FIG. 8 showed the percentages of the cells generating radicals ROS: about 40.3±10.2% for the bacteria treated with AgNP/SWN after 2 hours, and less than 10% for the bacteria treated with SWN.

According to the above assays, effects of the composite of the present invention in inhibiting bacteria were factually achieved by the radicals ROS generated by bacteria.

The present invention provides an composite of AgNPs which can effectively kill bacteria in lower silver ion concentrations, particularly the silver-resistant strains. The present invention also verifies that the composite kills the bacteria by radicals but not dissociated silver ions, so that side effects of the silver ions can be signifacantly decreased.

What is claimed is:

1. A composite of silver nanoparticles (AgNPs) and inorganic clay for inhibiting growth of silver-resistant bacteria, the composite comprising AgNPs and layered inorganic clay nanoparticles, wherein the composite has a particle size ranging from 5 nm to 100 nm, the layered inorganic clay has an aspect ratio (width/thickness ratio) of about 10 to 100,000 and serves as carriers of the AgNPs, the ratio of ionic equivalent of the AgNPs to cation exchanging equivalent (CEC) of the layered inorganic clay (Ag⁺/CEC) ranges from 0.1/1 to 200/1, and the AgNPs/clay weight ratio ranges from 1/99 to 99/1.

2. The composite of claim 1, wherein the silver-resistant bacteria are multi-silver-resistant bacteria.

3. The composite of claim 1, wherein the silver-resistant bacteria are silver-resistant Acinetobacter baumannii or Escherichia coli.

4. The composite of claim 1, wherein the layered inorganic clay has an aspect ratio of about 100 to 1,000.

5. The composite of claim 1, wherein the layered inorganic clay is bentonite, laponite, montmorillonite, synthetic mica, kaolin, talc, attapulgite clay, vermiculite or double hydroxide (LDH) nanoparticles.

6. The composite of claim 1, wherein the layered inorganic clay is nanosilicate platelets or bentonite.

7. The composite of claim 1, wherein the AgNPs/clay weight ratio is about 1/99 to 20/80.

8. The composite of claim 1, wherein the AgNPs/clay weight ratio is about 3/97 to 10/90.

9. A solution for inhibiting growth of silver-resistant bacteria, the solution comprising a solvent and a composite of silver nanoparticles (AgNPs) and inorganic clay nanoparticles, wherein the composite has a particle size ranging from 5 nm to 100 nm, the layered inorganic clay has an aspect ratio (width/thickness ratio) of about 10 to 100,000 and serves as carriers of the AgNPs, the ratio of ionic equivalent of the AgNPs to cation exchanging equivalent (CEC) of the layered inorganic clay (Ag⁺/CEC) ranges from 0.1/1 to 200/1, the AgNPs/clay weight ratio ranges from 1/99 to 99/1, and the composite is present in an amount of 0.0001 wt % to 10.0 wt % in the solution.

10. The solution of claim 9, wherein the composite is present in an amount of 0.001 wt % to 1.0 wt % in the solution.

11. The solution of claim 9, wherein the composite is present in an amount of 0.01 wt % to 0.2 wt % in the solution.

12. The solution of claim 9, wherein the layered inorganic clay has a cation exchanging equivalent (CEC) of about 0.1 mequiv/g to 5.0 mequiv/g.

13. The solution of claim 9, wherein the ratio (Ag⁺/CEC) ranges from 0.1/1 to 10/1.

14. The solution of claim 9, wherein the ratio (Ag⁺/CEC) ranges from 0.5/1 to 2/1.

15. The solution of claim 9, wherein the solvent is an organic solvent.

16. The solution of claim 9, wherein the solvent is water.

17. A method for inhibiting bacterial growth of silver-resistant bacteria, comprising a step of adding a composite of silver nanoparticles (AgNPs) and inorganic clay nanoparticles to silver-resistant bacteria, wherein the composite has a particle size ranging from 5 nm to 100 nm, the inorganic clay nanoparticle has an aspect ratio (width/thickness ratio) of about 10 to 100,000 and serves as carriers of the AgNPs, the ratio of ionic equivalent of the AgNPs to cation exchanging equivalent (CEC) of the layered inorganic clay (Ag⁺/CEC) ranges from 0.1/1 to 200/1, and the AgNPs/clay weight ratio ranges from 1/99 to 99/1.

18. The method of claim 17, wherein the ratio (Ag⁺/CEC) ranges from 0.5/1 to 2/1.

19. The method of claim 17, wherein the AgNPs/clay weight ratio is from 1/99 to 10/90.
